(19) United States
(12) Patent Application Publication
(10) Pub. No.: US 2022/0062169 A1
(43) Pub. Date: Mar. 3, 2022

(54) OCULAR LENS, PHARMACEUTICAL COMPOSITION, AND USES THEREOF

(71) Applicant: GoldRed Nanobiotech CO., LTD., Taoyuan City (TW)

(72) Inventors: Hong-Shong CHANG, Tao-Yuan (TW); Yun-Yu CHEN, New Taipei City (TW); Wei-Chung LAI, New Taipei City (TW); Juin-Hong CHERNG, Taipei City (TW); Kuan-Jung LI, Tainan City (TW)

(73) Assignee: GoldRed Nanobiotech CO., LTD., Taoyuan City (TW)

(21) Appl. No.: 17/423,115

(22) PCT Filed: Jan. 17, 2020
(86) PCT No.: PCT/CN2020/072718

§ 371 Date: Jul. 15, 2021

Related U.S. Application Data
(60) Provisional application No. 62/794,563, filed on Jan. 19, 2019.

(51) Int. Cl. A61K 9/00; A61K 9/51; A61K 33/242; A61P 27/02

(52) U.S. Cl. CPC A61K 9/0051; A61P 27/02; A61K 33/242; A61K 9/5123

(57) ABSTRACT

Disclosed herein are an ocular lens and a pharmaceutical composition. The ocular lens of the present disclosure is characterized in having a dihydrolipoic acid (DHLA) coated gold nanoclusters absorbed thereon. The pharmaceutical composition of the present disclosure comprises a DHLA coated gold nanocluster, and a pharmaceutically acceptable excipient. According to some embodiments of the present disclosure, the DHLA coated gold nanoclusters are capable of reducing intracellular ROS levels, promoting tissue repair, and inhibiting pathological angiogenesis. Accordingly, also disclosed herein are methods of treating ocular conditions by uses of the present contact lens or pharmaceutical composition.

### CROSS-REFERENCE TO RELATED APPLICATIONS

This application relates to and claims the benefit of U.S. Provisional Application No. 62/794,563, filed Jan. 19, 2019; the content of the application is incorporated herein by reference in its entirety.

### BACKGROUND OF THE INVENTION

### 1. Field of the Invention

The present disclosure in general relates to the field of disease treatment. More particularly, the present disclosure relates to the treatment of ocular conditions by use of a contact lens having dihydrolipoic acid (DHLA) coated gold nanoclusters absorbed thereon, or a pharmaceutic composition containing DHLA coated gold nanoclusters.

### 2. Description of Related Art

Reactive oxygen species (ROS) is a chemically reactive radical derived from molecular oxygen. Exemplary ROS includes, hydrogen peroxide (H₂O₂), superoxide (O₂⁻), hydroxyl radical (⁻OH), singlet oxygen (^{I}[O₂]), and alpha-oxygen (α-O). ROS is produced as a nature product during metabolic process, and plays an important role in cell signaling and homeostasis. However, it is known that that excessive ROS would induce oxidative stress resulting in cellular damage and ultimately cellular death.

ROS has been implicated in the development and/or progress of various ocular conditions, including dry eye, conjunctivitis, uveitis, keratitis, retinitis, cataract, refractive error, glaucoma, optic neuropathy, macular degeneration, retinopathy, and retinitis pigmentosa. Unfortunately, some ocular conditions are still incurable today in spite of the enormous progress of science and research in this area in recent years.

In view of the foregoing, there exists in the related art a need for a method of reducing the ROS level of the eyes thereby efficiently treating ocular conditions.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a ocular lens for the treatment of ocular conditions. According to embodiments of the present disclosure, the ocular lens is characterized in having a dihydrolipoic acid (DHLA) coated gold nanocluster absorbed thereon. In structure, the DHLA coated gold nanocluster consists of, a gold nanocluster formed by a plurality of gold nanoparticles, and a plurality of DHLAs coated on the gold nanocluster. Preferably, the DHLA coated gold nanocluster has a particle size of about 0.1 to 20 nm.

According to some embodiments of the present disclosure, the ocular lens is prepared by incubating a contact lens with a solution of the DHLA coated gold nanocluster having a concentration of 1-100 nM and pH value of 7.5-9.0 at 15-50° C. for at least 30 minutes. Preferably, the DHLA coated gold nanocluster solution is subjected to a pressure of 1.2-2.0 atmosphere (atm; equivalent to 912-1,520 mm Hg, or 1.2×10⁵-2×10⁵ Pa) at 120-140° C. for 10-60 minutes prior to the incubation step. According to certain working examples of the present disclosure, the contact lens is incubated with 10-50 nM of the DHLA coated gold nanocluster solution.

Also disclosed herein is a kit for treating an ocular condition. The kit comprises a solution containing 1-100 nM of the DHLA coated gold nanocluster, and a contact lens immersed in the solution. According to some preferred embodiments, the DHLA coated gold nanocluster solution of the present kit is pre-treated by a pressure of 1.2-2.0 atm at 120-140° C. for 10-60 minutes.

Another aspect of the present disclosure is directed to a pharmaceutical composition comprising a DHLA coated gold nanocluster, and a pharmaceutically acceptable excipient. Preferably, the DHLA coated gold nanocluster solution of the present pharmaceutical composition is pre-treated by a pressure of 1.2-2.0 atm at 120-140° C. for 10-60 minutes.

The present disclosure also provides a method of treating an ocular condition in a subject by use of the ocular lens or the pharmaceutical composition in accordance with any aspect or embodiment of the present disclosure. According to some embodiments, the method comprises placing the present ocular lens onto the cornea of the subject thereby ameliorating or alleviating the symptoms associated with the ocular condition. According to certain embodiments, the method comprises administering to the eye of the subject an effective amount of the present pharmaceutical composition thereby ameliorating or alleviating the symptoms associated with the ocular condition.

The ocular condition treatable with the present ocular lens and/or pharmaceutical composition may be any ocular condition associated with and/or caused by injury, ROS or angiogenesis; for example, eye injury, dry eye, conjunctivitis, uveitis, keratitis, retinitis, cataract, refractive error, glaucoma, optic neuropathy, macular degeneration, retinopathy, or retinitis pigmentosa.

In the present disclosure, the subject is a mammal; preferably, a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1A is a histogram depicting the anti-oxidative activity of the present DHLA coated gold nanoclusters according to Example 1 of the present disclosure, in which the fluorescence intensity of endothelial progenitor cells (EPCs) administered with specified treatment was measured by flow cytometry.

FIG. 1B is a line chart depicting the anti-oxidative activity of the present DHLA coated gold nanoclusters according to Example 1 of the present disclosure, in which the absorbance at 485 nm and 539 nm of the bovine cornea epithelial cells (BCEs) treated with DHLA coated gold nanoclusters at specified concentrations and irradiated with or without blue light were analyzed by spectrometer.

FIG. 2A is a histogram depicting the anti-oxidative activity of the present DHLA coated gold nanoclusters according to Example 1 of the present disclosure, in which the DHLA coated gold nanoclusters with or without sterilization pre-treatment were administered to EPCs for one hour, and the fluorescence intensity of EPCs was measured by flow cytometry. TBHP: t-butyl hydroperoxide, serving as the positive control. R: contact lens solution only. *, p<0.05; **, p<0.01; n=3.

FIG. 2B is a line chart depicting the anti-oxidative activity of the present DHLA coated gold nanoclusters according to Example 1 of the present disclosure, in which the absorbance at 485 nm and 539 nm of the BCEs treated with sterilized DHLA coated gold nanoclusters at specified concentrations and irradiated with or without blue light were analyzed by spectrometer.

FIG. 2C is a histogram depicting the percentage (%) of reduced ROS level according to Example 1 of the present disclosure, in which the ROS levels in BCEs treated with sterilized and non-sterilized DHLA coated gold nanoclusters at specified concentrations were respectively measured thereby determining the percentage of reduced ROS level.

FIG. 2D is a histogram depicting the ROS inhibition ratio of sterilized and non-sterilized DHLA coated gold nanoclusters according to Example 1 of the present disclosure, in which the BCEs treated with 1,000 nM sterilized or non-sterilized DHLA coated gold nanoclusters were exposed to blue light irradiation for 10, 20, or 30 minutes. The absorbance at 485 nm and 539 nm of the BCEs was measured by spectrometer thereby determining the ROS inhibitory activities of sterilized and non-sterilized DHLA coated gold nanoclusters.

FIG. 3 is a histogram depicting the absorption level of specified gold nanoclusters on contact lens according to Example 2 of the present disclosure.

FIGS. 4A and 4B are histograms respectively depicting the GSH expression level of cells treated with specified treatment according to Example 2 of the present disclosure. Buffer_0 nM: medium only; Buffer_30 nM: medium containing 30 nM DHLA coated gold nanoclusters; Lens_0 nM: contact lens pre-treated with medium; Lens_30 nM: contact lens pre-treated with medium containing 30 nM DHLA coated gold nanoclusters. ***, p<0.001.

FIGS. 5A to 5C are histograms depicting the GSH expression level of cells treated with specified treatment according to Example 2 of the present disclosure. Buffer_0 nM: medium only; Buffer_30 nM: medium containing 30 nM DHLA coated gold nanoclusters; Lens_0 nM: contact lens pre-treated with medium; Lens_30 nM: contact lens pre-treated with medium containing 30 nM DHLA coated gold nanoclusters. *, p<0.05.

FIGS. 6A and 6B are photographs of wound healing assay according to Example 3 of the present disclosure. PBS treatment: cells treated with phosphate-buffered saline (PBS); R treatment: cells treated with eye drops; R+30F treatment: cells treated with eye drops containing 30 nM DHLA coated gold nanoclusters; R+50F treatment: cells treated with eye drops containing 50 nM DHLA coated gold nanoclusters; R+100F treatment: cells treated with eye drops containing 100 nM DHLA coated gold nanoclusters; R+200F treatment: cells treated with eye drops containing 200 nM DHLA coated gold nanoclusters.

FIGS. 7A to 7C are photographs depicting the corneal wound healing of mice administered with specified treatments according to Example 3 of the present disclosure. FIG. 7A: mechanical scrape model. FIG. 7B: STZ-induced model. FIG. 7C: oxygen-induced retinopathy (OIR) model.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “monomer” as used herein refers to both a molecule comprising one or more polymerizable functional groups prior to polymerization, and a repeating unit of a polymer. More specifically, the term “monomer” refers to any molecule that can be polymerized, that is, linked together via a chemical reaction to form a higher molecular weight species. A copolymer is said to comprise two or more different monomers.

As used herein, the term “crosslinking agent” refers to a compound having the ability to form stable covalent bonds. More specifically, the term “crosslinking agent” includes any molecule or atom that is capable of forming one or more crosslinks between molecules of the crosslinkable polymer and/or between two or more atoms in a single molecule of the crosslinkable polymer. The term “crosslink” as used herein refers to a covalent bond that links one polymer chain to another.

As used herein, the term “intraocular lens” should be given the broadest possible meaning within this context. In general, the term “intraocular lens” refers to a lens that is implanted into the interior of an eye to either replace the eye's natural lens or to otherwise augment vision regardless of whether or not the natural lens is removed. Intracorneal lenses and phakic lenses are examples of lenses that may be implanted into the eye without removal of the natural lens.

The term “contact lens” is art recognized and is intended to include those devices generally used for correction of visual acuity, for cosmetic purposes and for protection of the cornea, e.g., a device which does not correct for visual acuity. Contact lenses include those which are considered “hard”, e.g. poly(methyl methacrylate) (PMMA), which has excellent biocompatibility but has poor oxygen permeability; “gas-permeable”, e.g., poly(silicone methacrylate), which has excellent biocompatability and allows diffusion of oxygen through the polymeric structure; and “soft”, e.g., poly(hydroxyethyl methacrylate), which has excellent biocompatibility and also allows diffusion of oxygen through the polymeric structure by aqueous transport. Examples of materials used in contact lenses include, but are not limited to, poly(methyl methacrylate), poly(silicone acrylate), poly(silicone methacrylate), poly(fluoroacrylate), poly(fluoromethacrylate), poly(flurosilicone eacrylate), poly(silicone methacrylate), polymethacrylate, polyacrylate, polyurethane, poly(silicone urethane), polyitaconate, and a combination thereof. These polymeric materials can also be crosslinked by one or more crosslinking agents. A contact lens may generally include a convex surface and a concave surface configured for contacting an eye. Generally, a wide variety of contact lenses are known to those skilled in the art.

As used herein, a “pharmaceutically acceptable” component is one that is suitable for use with humans and/or animals without adverse side effects (such as toxicity, irritation and/or allergic response) commensurate with a reasonable benefit/risk ratio.

The term “treating” or “treatment” as used herein is intended to mean obtaining a desired pharmacological and/or physiologic effect, e.g., delaying or inhibiting the development or progression of an ocular condition. The effect may be prophylactic in terms of completely or partially preventing an ocular condition or the symptom thereof, and/or be therapeutic in terms of a partial or complete cure for an ocular condition and/or adverse effect attributable to the condition. The term “treating” or “treatment” as used herein includes preventative (e.g., prophylactic), curative or palliative treatment of an ocular condition in a mammal, particularly human; and includes: (1) preventative (e.g., prophylactic), curative or palliative treatment of an ocular condition from occurring in an individual who may be pre-disposed to the condition but has not yet been diagnosed as having it; (2) inhibiting an ocular condition (e.g., by arresting its development or progression); or (3) relieving an ocular condition (e.g., reducing symptoms associated with the condition). In general, the term “treating” or “treatment” as used herein refers to the reduction or resolution of an ocular condition (for example, the condition associated with and/or caused by ROS, angiogenesis, or ocular injury or damage), and/or to promote healing of injured or damaged ocular tissue.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/Kg). Alternatively, the effective amount can be expressed in the concentration of the active component (e.g., the DHLA coated gold nanocluster of the present disclosure), such as molar concentration, mass concentration, volume concentration, molality, mole fraction, mass fraction and mixing ratio. Persons having ordinary skills could calculate the human equivalent dose (HED) for the medicament (such as the DHLA coated gold nanocluster of the present disclosure) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “subject” refers to a mammal including the human species that is treatable with the ocular lens or pharmaceutic composition of the present invention. The term “subject” is intended to refer to both the male and female gender unless one gender is specifically indicated.

### II. Description of the Invention

(i) Ocular Lens

The first aspect of the present disclosure is directed to an ocular lens, which exhibits vision-correcting and/or anti-oxidative effects, thereby providing a means to treat ROS-associated ocular conditions and/or protect the eye from oxidative damage. Also disclosed herein are kits and methods for preparing the ocular lens of the present disclosure, and methods for treating ocular conditions, especially ocular conditions caused by and/or associated with ROS, by use of the present ocular lens.

The present ocular lens is characterized in having a DHLA coated gold nanocluster dispersed thereon in and/or absorbed thereon. The DHLA coated gold nanoclusters used in the present disclosure are known to the skilled practitioner as well as the process for their production (Lin et al., 2009, ACS Nano 3: 395-401); hence no further explanations are necessary with respect to their preparation. In structure, each DHLA coated gold nanocluster consists of, a gold nanocluster formed by a plurality of gold nanoparticles, and a plurality of DHLAs coated on the gold nanocluster. The DHLA coated gold nanoclusters have a fluorescent emission at 650 nm under an excitation wavelength at approximately 420 nm, hence will emit wavelength ranged from red to near infrared. Each gold nanocluster has a particle size of 0.1 to 20 nm, preferably from 1 to 15 nm, and more preferably from 2 to 13 nm. The dimension discussed above related to the gold nanoparticle of the present disclosure is in dried state; however, it is of advantage if the gold nanocluster used in the present disclosure is water-soluble or at least dispersible in aqueous medium and/or water; the hydrodynamic size of the dried nanocluster can be significantly larger than the dried size due to the coupling of surrounding solvent molecule such as water. In one specific embodiment example, the gold nanocluster has a hydrodynamic size corresponds to 1 to 30 kDa polyethylene glycol (PEG).

Depending on intended purposes, the ocular lens of the present disclosure may be prepared into the form of an intraocular lens or a contact lens (e.g., soft contacts lens, hard contact lens, and gas-permeable contact lens). During the manufacturing process, the present ocular lens is incubated with a solution of the DHLA coated gold nanocluster having a concentration of 1-100 nM and pH value of 7.5-9.0 (e.g., pH 7.5, 7.6, 7.7, 7.8, 7.9, 8.0, 8.1, 8.2, 8.3, 8.4, 8.5, 8.6, 8.7, 8.8, 8.9, or 9.0) at 15-50° C. (e.g., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.) for at least 30 minutes (for example, 30, 31, 32, 33, 34, 35, 36, 37, 38, 38, 40, 41, 42, 43, 44, 45, 46, 47, 48, 49, 50, 51, 52, 53, 54, 55, 56, 57, 58, 59 or 60 minutes, or longer), so that the DHLA coated gold nanocluster would be absorbed on the surface of the ocular lens and/or dispersed in the structure of the ocular lens. According to some working examples, the present ocular lens is incubated with a solution of the DHLA coated gold nanocluster having a pH value of 7.5-8.5 at 20-45° C. for 30 minutes. In one specific example, the present ocular lens is incubated with a solution of the DHLA coated gold nanocluster having a pH value of 8.0-8.5 at 20-40° C. for 30 minutes.

Optionally, the DHLA coated gold nanocluster solution is pre-treated by 1.2-2.0 atm (e.g., 1.2, 1.3, 1.4, 1.5, 1.6, 1.7, 1.8, 1.9, or 2.0 atm) at 120-140° C. (e.g., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., or 140° C.) for 10-60 minutes (e.g., 10, 15, 20, 25, 30, 35, 40, 45, 50, 55 or 60 minutes). Preferably, the DHLA coated gold nanocluster solution is pre-treated by 1.2-1.8 atm at 125-135° C. for 20-50 minutes. More preferably, the DHLA coated gold nanocluster solution is pre-treated by 1.2-1.5 atm at 130-135° C. for 20-40 minutes. According to one specific example, the DHLA coated gold nanocluster solution is pre-treated by 1.5 atm at 132° C. for 30 minutes.

According to some working examples of the present disclosure, compared with the DHLA coated gold nanocluster solution without pre-treatment, the DHLA coated gold nanocluster solution pre-treated by specified conditions exhibits higher absorption capability and anti-oxidation ability. The ocular lens may be incubated with 1-100 nM of the pre-treated DHLA coated gold nanocluster solution at 20-40° C. for 30 minutes so as to absorb the anti-oxidative DHLA coated gold nanocluster on the surface and/or in the structure thereof; for example, incubated with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nM of the pre-treated DHLA coated gold nanocluster solution. Preferably, the ocular lens is incubated with 10-50 nM of the pre-treated DHLA coated gold nanocluster solution. According to one specific example, the ocular lens incubated with 30 nM provides an protective effect on the cornea cell against blue light irradiation.

The structure of the ocular lens is familiar with the person having ordinary skill in the art. For example, the ocular lens may comprise at least one monomer, and a crosslinking agent for polymerizing the at least one monomer.

In practice, the manufacture (e.g., the shape, structure, central axis, thickness, and etc.) of the ocular lens may vary with intended use, and may be performed by any method commonly used or known in the art. For example, the intraocular lens can be manufactured via thermal polymerization method or photopolymerization method. In this case, the monomer for manufacturing the intraocular lens may be propylene, ester, siloxane, acrylamide, imide, hydrophobic or hydrophilic acrylic, or other biocompatible materials. Exemplary monomers commonly used to manufacture the intraocular lens include, but are not limited to, methyl methacrylate (MMA), butyl methacrylate, hydroxyethyl methacrylate (HEMA), cyclohexyl methacrylate, glycerol methacrylate, dimethylacrylamide, methacrylic acid, 2-phenoxyethyl acrylate, 2-phenylethylthio acrylate, 2-phenylethylamino acrylate, phenyl acrylate, benzyl acrylate, 2-phenylethyl acrylate, 3-phenylpropyl acrylate, 3-phenoxypropyl acrylate, 4-phenylbutyl acrylate, 4-phenoxybutyl acrylate, 4-methylphenyl acrylate, 4-methylbenzyl acrylate, 2-2-methylphenylethyl acrylate, 2-3-methylphenylethyl acrylate, 2-4-methylphenylethyl acrylate, pyrrolidone (NVP), silicone acrylate, silicone methacrylate, fluoroacrylate, fluoromethacrylate, flurosilicone eacrylate, silicone methacrylate, methacrylate, acrylate, urethane, silicone urethane, itaconate, trifluoroethyl methacrylate, hexafluoroisopropyl methacrylate, perfluorooctylethyloxypropyl ene methacrylate, vinyl alcohol (VA), vinylacetate, ethylene glycol, propylene glycol, perfluoropolyether, and a combination thereof. The silicone component listed above may be selected from the group consisting of, monomethacrylate terminated poly dim ethyl siloxane, bis-3-acryloxy-2-hydroxypropyloxypropyl polydialkylsiloxane, mono-(3-methacryloxy-2-hydroxypropyloxy)propyl terminated polydialkylsiloxane, mono-butyl terminated polydialkylsiloxane, and a combination thereof.

Regarding the contact lens, it may be manufactured by lathe method or molding method. Examples of the monomer suitable for manufacturing the contact lens include, but are not limited to, N-vinyl-N-methyl acetamide (VMA), pyrrolidone (NVP), 1,4-butanediol vinyl ether (BVE), ethylene glycol vinyl ether (EGVE), diethylene glycol vinyl ether (DEGVE), 1,4-cyclohexanedimethanol vinyl ether (CHDMVE), methyl methacrylate (MMA), 2-hydroxybutyl methacrylate (HOB), 2-ethylhexy methacrylate (EHMA), tert-butyl methacrylate (tBMA), N,N-dimethylacrylamide (DMA), hydroxyethyl methacrylate (HEMA), ethoxyethyl methacrylamide (EOEMA), ethylene glycol methyl ether methacrylate (EGMA), isobornyl methacrylate (IBM), glyceryl methacrylate (PGMA), silicone acrylate, silicone methacrylate, fluoroacrylate, fluoromethacrylate, flurosilicone eacrylate, silicone methacrylate, methacrylate, acrylate, urethane, silicone urethane, itaconate, vinyl alcohol (VA), and a combination thereof. The silicone component listed above may be selected from the group consisting of, monomethacrylate terminated poly dim ethyl siloxane, bis-3-acryloxy-2-hydroxypropyloxypropyl polydialkylsiloxane, mono-(3-methacryloxy-2-hydroxypropyloxy)propyl terminated polydialkylsiloxane, mono-butyl terminated polydialkylsiloxane, and a combination thereof. According to certain embodiments, the monomer is HEMA.

Exemplary crosslinking agents for polymerizing the monomer(s) during the manufacturing process include, but are not limited to, ethylene glycol diacrylate (EGDA), 1,6-hexanediol diacrylate (HDODA), ethylene, glycol, dimethacrylate, diethylene glycol dimethacrylate (DEGDMA), trimethylolpropane (TMP), trimethacrylate (TA), trimethylolpropane trimethacrylate (TMPTMA), allyl methacrylate (AMA), divinylbenzene (DVB), pentaerythritol tetramethacrylate (PETMA), trimethylolpropane trimethacrylate (TMPTA), ethylene glycol dimethacrylate (EGDMA), 1,3-propanediol dimethacrylate, 1,6-hexanediol dimethacrylate, 1,4-butanediol dimethacrylate, 1,4-butanediol diacrylate, allyl acrylate, and a combination thereof.

As would be appreciated, in addition to the intraocular or contact lens, the present DHLA coated gold nanocluster may also be applied to the spectacle lens, for example, being coated on or absorbed to the surface of the spectacle lens.

Another aspect of the present disclosure is directed to a kit for the preparation of the present ocular lens. The kit comprises a solution containing the DHLA coated gold nanocluster, and a contact lens or an intraocular lens immersed in the solution. In general, the DHLA coated gold nanocluster is present in the solution in a concentration of 1-100 nM, and the solution may be any solution suitable for preserving the contact lens or intraocular lens, for example, the solution containing sodium chloride, boric acid, sodium borate, polylysine (PS), polyphosphoric acid, polyvinylpyrrolidone (PVP), ethylenediaminetetraacetic acid (EDTA), poloxamer, and/or antibacterial agent.

Also disclosed herein is a method of treating an ocular condition in a subject in need thereof by use of the present ocular lens. In general, the ocular condition treatable with the present ocular lens may be caused by and/or associated with injury, for example, a blow to the eye with a baseball, rock or other hard objects that damages the eye, eyelids, and muscles or bone surrounding the eye; a cut or scratch by a stick, finger, wood chip, metal shaving, sand or glass that damages the cornea; a chemical burn by soap, shampoo, or other chemical reagents that cause serious burns inside the eye; or a radiation by blue light, ultraviolet (UV) or X-ray that damages to the cornea and retina. According to certain embodiments, the ocular condition is caused by and/or associated with ROS or oxidative stress that results in cellular death by damaging lipids, proteins, carbohydrates, and nucleic acids (e.g., deoxyribonucleic acid (DNA)). According to some embodiment, the ocular condition is caused by and/or associated with injury, which leads to corneal damage. According to alternative embodiments, the ocular condition is caused by and/or associated with angiogenesis, such as the ocular angiogenesis (OA) occurs in retina choroid and/or cornea that leads to a broad spectrum of disorders, including age-related macular degeneration (AMD), diabetic retinopathy, retinal artery or vein occlusion, retinopathy of prematurity (ROP), neovascular glaucoma, and corneal neovascularization. Non-limiting examples of ocular condition treatable with the present ocular lens include, eye injury, dry eye, conjunctivitis, uveitis, keratitis, retinitis, cataract, refractive error, glaucoma, optic neuropathy, macular degeneration, retinopathy, and retinitis pigmentosa.

In some embodiments, the ocular lens of the present disclosure is useful in treating cataract or refractive error in a subject, in which the ocular lens is manufactured into the form of an intraocular lens that can be transplanted into the eye of the subject thereby improving his/her vision, as well as reducing/eliminating the oxidative stress in the eye.

In alternative embodiments, the ocular lens of the present disclosure is manufactured into the form of a contact lens that can be placed onto the cornea of a subject in need thereof so as to achieve the vision-correcting and/or therapeutic (e.g., anti-oxidative, anti-angiogenic, or promoting tissue healing) effects.

(ii) Pharmaceutical Composition

Another aspect of the present disclosure pertains to a pharmaceutical composition comprising a DHLA coated gold nanocluster as mentioned in section (i) of the present disclosure, and a pharmaceutically acceptable excipient.

Optionally, the DHLA coated gold nanocluster of the present pharmaceutical composition is pre-treated by a pressure of 1.2-2.0 atm at 120-140° C. for 10-60 minutes, followed by mixing with the pharmaceutically acceptable excipient.

Depending on desired purposes, the DHLA coated gold nanocluster is present in the pharmaceutical composition at a level of about 0.01% to 99.9% by weight, based on the total weight of the pharmaceutical composition. In some embodiments, the DHLA coated gold nanocluster is present at a level of at least 0.1% by weight, based on the total weight of the pharmaceutical composition. In certain embodiments, the DHLA coated gold nanocluster is present at a level of at least 5% by weight, based on the total weight of the pharmaceutical composition. In still other embodiments, the DHLA coated gold nanocluster is present at a level of at least 10% by weight, based on the total weight of the pharmaceutical composition. In still yet other embodiments, the DHLA coated gold nanocluster is present at a level of at least 25% by weight, based on the total weight of the pharmaceutical composition.

The pharmaceutically acceptable excipient suitable for formulating the present pharmaceutical composition may be any ophthalmically acceptable excipient known by the person having ordinary skill in the art, for example, an ophthalmic buffer (such as phosphate buffer (e.g., sodium dihydrogen phosphate and disodium hydrogen phosphate), borate buffer (e.g., boric acid or the salt thereof), citrate buffer (e.g., citric acid or the salt thereof), or a combination thereof), a chelating agent (such as disodium edetate, trisodium edetate, tetrasodium edetate, or a combination thereof), tonicity agent (such as propylene glycol, diethylene glycol, triethylene glycol, glycerol, dextrose, glycerin, mannitol, potassium chloride, sodium chloride, or a combination thereof), viscosity or suspending agent (such as methyl cellulose, ethyl cellulose, hydroxyethylcellulose, polyethylene glycol, carboxymethyl cellulose, hydroxypropylmethyl cellulose, cross-linked acrylic acid polymer, or a combination thereof), pH modifying agent (such as mineral acid, potassium hydroxide, sodium hydroxide, hydrochloric acid, or a combination thereof), or a combination thereof.

Optionally, the pharmaceutical composition of this invention may further include a therapeutic agent known to prevent, alleviate, or ameliorate the symptoms of ocular conditions. For example, the therapeutic agent may be an anti-inflammatory agent (e.g., cyclosporine, or corticosteroid), a tear-stimulating drug or artificial tears (e.g., cholinergics (such as pilocarpine, cevimeline), or hydroxypropyl cellulose), an anti-infectious agent (e.g., an anti-fungal agent, an anti-bacterial agent, or an anti-viral agent agent), an anti-oxidant (e.g., lutein, zeaxanthin, vitamin A, vitamin C, vitamin E, omega-3 fatty acid, selenium, or zinc), an anti-angiogenic agent (e.g., the inhibitor of vascular endothelial growth factor (VEGF) or its receptor (VEGFR), or the inhibitor of platelet-derived growth factor (PDGF) or its receptor (PDGFR)).

Also disclosed herein is a method of treating an ocular condition in a subject by use of the present pharmaceutical composition. The method comprises administering to the eye of the subject an effective amount of the present pharmaceutical composition thereby alleviating or ameliorating the symptoms associated with the ocular condition.

As described in section (i) of the present disclosure, the ocular condition treatable with the present pharmaceutical composition may be caused by and/or associated with injury, ROS or angiogenesis; for example, eye injury, dry eye, conjunctivitis, uveitis, keratitis, retinitis, cataract, refractive error, glaucoma, optic neuropathy, macular degeneration, retinopathy, and retinitis pigmentosa.

In general, the subject treatable with the present ocular lens, pharmaceutical composition and/or method is a mammal, for example, a human, mouse, rat, rabbit, monkey, chimpanzee, dog, cat, pig, horse, pig, goat, or sheep. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example

Materials and Methods

Preparation of DHLA Coated Gold Nanoclusters

Fluorescent gold nanoclusters used in this study were prepared as previously described (Lin et al., ACS Nano, 2009, 3: 395-401). Briefly, 6-nm gold nanoparticles stabilized with didodecyldimethylammonium bromide (AuNP@DDAB) were synthesized via an established single-phase reaction (Jana and Peng, J Am Chem Soc., 2003, 125:14280-14281). Subsequent further dropwise addition of gold precursor solution (AuCl₃ in DDAB-toluene solution) caused a gradual loss of plasmon absorption until the solution turned yellowish transparent. Ligand exchange was performed by adding the as-prepared nanoclusters to the reduced lipoic acid (DHLA, dihydrolipoic acid), which was freshly reduced by tetrabutylammonium borohydride (TBAB) with a molar ratio of lipoic acid to TBAB=4:1. This leads to dark-brown nanocluster agglomerates in the resulting mixture, and additional UV lamp exposure (365 nm, 30 mins) was treated to condense the agglomerates. After discarding the supernatant, nanoclusters were re-dispersed in methanol and precipitated again in additional chloroform to remove free surfactants. The dried nanoclusters precipitate could be dispersed in borate buffer (pH 9). Further purification was achieved by three runs of ultracentrifugation (110,000 rpm) to remove excess DHLA. Gold nanoclusters were collected, and the PBS buffer was changed through a centrifuge filter of 30 kDa molecular weight cut-off (MWCO), leading to a colloidally stable transparent solution of NCs without plasmon peak. The concentration of gold nanoclusters was measured by the extinction coefficient of about 450,000 M⁻¹ cm⁻¹ at 420 nm.

For the purpose of enhancing the absorption capability and anti-oxidation ability, the DHLA coated gold nanoclusters were sterilized under a pressure of 1.5 atm at 132° C. for 30 minutes.

Anti-Oxidative Activity—Antimycin A (AA) Stimulation

To evaluate the effect of the present gold nanoclusters on AA-induced ROS, endothelial progenitor cells (EPCs) were seeded into 96-well culture plate (2×10⁴ cells/well). 24 hours later, 40 μl of DHLA coated gold nanoclusters diluted in culture medium were added to the EPCs at a final concentration of 7.5, 15 or 25 nM, followed by incubating at 37° C. for 16 hours. After replacing the culture medium with fresh medium, AA (a mitochondrial ionophore for ROS release; final concentration: 50 μM) was added to the EPCs, and incubated at 37° C. for 1 hour. The EPCs were harvested followed by subjecting to flow cytometry analysis of fluorescence intensity, which was positively correlated with intracellular ROS level.

Anti-Oxidative Activity—Blue Light Irradiation

On day one, bovine cornea epithelial cells (BCEs) were seeded into a 96-well culture plate (2×10⁴ cells/well). 24 hours later, 40 μl of DHLA coated gold nanoclusters diluted in culture medium were added to the BCEs at a final concentration of 0.2, 2, 5, 10, 20, 40, 80, 160, 250, 500, 1,000 or 2,000 nM, followed by incubating at 37° C. for 16 hours. After removing the culture medium, the fluorogenic probe 2′, 7′-dichlorodihydrofluorescein diacetate (DCF-DA) was added to each well. The BCEs were incubated at 37° C. for 1 hour, and then exposed to blue light irradiation (wavelength: 460-465 nm) at 37° C. for 0, 10, 20 or 30 minutes. The cellular absorbance at wavelengths of 485 nm and 539 nm were measured by spectrometer. The ratio of 485 nm to 539 nm (i.e., 485/539 nm) was positively correlated with intracellular ROS level.

The percentage (%) of the reduced ROS level is calculated by the equation of, Reduced level of ROS (%)=[(ROS level of irradiated cells treated with sterilized DHLA coated gold nanocluster)/ROS level of irradiated cells)]−[(ROS level of irradiated cells treated with non-sterilized DHLA coated gold nanocluster)/ROS level of irradiated cells)].

Determining Absorption Capability of DHLA coated Gold Nanoclusters

To determine the absorption capability of DHLA coated gold nanoclusters, the contact lens were incubated with a solution (pH 8.0-8.5) at 20-40° C. under 1 atm (normal atmospheric condition; about 760 mm Hg, or 1×10⁵ Pa) for 30 minutes, wherein the solution contained 15 nM, 25 nM or 35 nM DHLA coated gold nanoclusters with or without sterilization pre-treatment as mentioned above (i.e., being sterilized under a pressure of 1.5 atm at 132° C. for 30 minutes). The sample was then soaked in 20% (v/v) nitric acid solution. After adding 1 ml of high-purity aqua regia and incubating at room temperature for one day to release the DHLA coated gold nanoclusters from the contact lens, 9 ml deionized water was added. The concentration of the DHLA coated gold nanoclusters in the mixture was then analyzed by inductively coupled plasma mass spectrometry (ICP-MS). 10 ppm (HIGH-PURITY) Au standard solution was diluted to a concentration of 0.8 ppb, 4 ppb and 20 ppb standard solution with 1% aqua regia (v/v) to establish a calibration plot. The absorption capability of DHLA coated gold nanoclusters was determined from the calibration plot based on the concentration analyzed by ICP-MS.

GSH Detection

Two models were used in this study to examine the effect of contact lens having DHLA coated gold nanoclusters absorbed thereon on cornea cells. In the defense model, 5×10⁵ BCEs were seeded on a contact lens. 16 hours later, the contact lens was immersed in a solution containing 30 nM DHLA coated gold nanoclusters, and simultaneously exposed to a blue light irradiation for 24 hours. In the repair model, 5×10⁵ BCEs seeded on a contact lens were exposed to a blue light irradiation for 24 hours followed by immersing in a solution containing 30 nM DHLA coated gold nanoclusters for 16 hours.

The expression of glutathione (GSH) in both models was determined by dyes to estimate cellular levels. In brief, after removing the culture medium, the cells were washed by 100 μl PBS. 100 μL prewarmed dye diluted in PBS was then added to the cells followed by incubating at 37° C. for 30 minutes. The images of cells were captured by fluorescence microscope, and the fluorescence excitation and emission wavelength at 404 and 526 nm were measured.

Wound Healing Assay

Wound healing assay was used to determine the effect of the present DHLA coated gold nanoclusters on tissue repair. In brief, rabbit cornea epithelial cells were cultured to confluence in a culture dish. After washing with PBS, a scratch wound was created on the cells by a pipette tip. Specified treatments (including PBS, and commercial eye drops containing 0, 30, 50, 100, or 200 nM DHLA coated gold nanoclusters) were added to the cells. 15 minutes later, the medium was replaced with low serum medium containing 1% fetal bovine serum (FBS). After culturing at 37° C. for 24 hours, images of cells administered with specified treatments were taken by optical microscope, and the distance of the scratched area was calculated by software.

Animal Model

Three animal models were used in this study to evaluate the tissue repairing activity of the present gold nanoclusters.

In the mechanical scrape model, eight-week-old female C57BL/6 mice were anesthetized by an intraperitoneal injection of a mixture of zoletil (6 mg/kg) and xylazine (3 mg/kg). One filter paper (0.9 mm diameter) soaked with 20% ethanol was placed on the central cornea of right eye for one minute and then irrigated extensively with PBS. Subsequently, mechanical epithelial scrape was performed using a punch under a dissection microscope to create a circular injury (2 mm diameter) at the entire corneal region of the mouse eye without encroaching the corneal stroma, limbus or conjunctiva. 50 μl of the present DHLA coated gold nanoclusters (diluted in PBS, concentration: 200 μM were added to the right eye one day after the scrape injury. In the control group, right eye was treated with 50 μl of PBS. Wound size was determined by staining with topical fluorescein, and photographed with a digital camera. The area of defect was quantified from the photographs using a computer-assisted image analyzer, and was calculated as the percentage of residual epithelial defect at each time point/initial wound area.

In the streptozotocin (STZ)-induced model, 65 mg/kg STZ was intraperitoneally injected into male Brown Norway (BN) rats for 3 days. On day 4, the present DHLA coated gold was freshly dissolved in 0.01 M sodium citrate (concentration: 200 μM), and intravenously injected to the STZ treated mice (2 ml/kg) via the tail vein. Sham rats received only the vehicle (0.01 M sodium citrate) in a volume of 2 ml/kg intravenously via the tail vein. Then, the mechanical scrape model was performed and with or without DHLA coated gold nanoclusters were added for the wound healing acceleration test as the same method describing above.

In the oxygen-induced retinopathy (OIR) model, 1,000 μl of the present DHLA coated gold nanoclusters (diluted in PBS, concentration: 1 μM) were intraperitoneally injected into newly born C57BL/6 mice (6-days old) under room air (20.8% oxygen). In the control group, the mice were treated with 1,000 μl of PBS. One day later, the mice were exposed to high levels of oxygen (75%) for 5 days and then returned to room air (20.8% oxygen). The changes in oxygen levels induced a relative hypoxia in the animals to which the endothelial cells responded by activating oxygen-responsive elements, such as the hypoxia inducible factor-1 (HIF-I) and VEGF. Five days later, the eyes were enucleated and examined to determine the level of neovascularization by histological analysis.

### Example 1 Anti-oxidative Activity of DHLA Coated Gold Nanoclusters

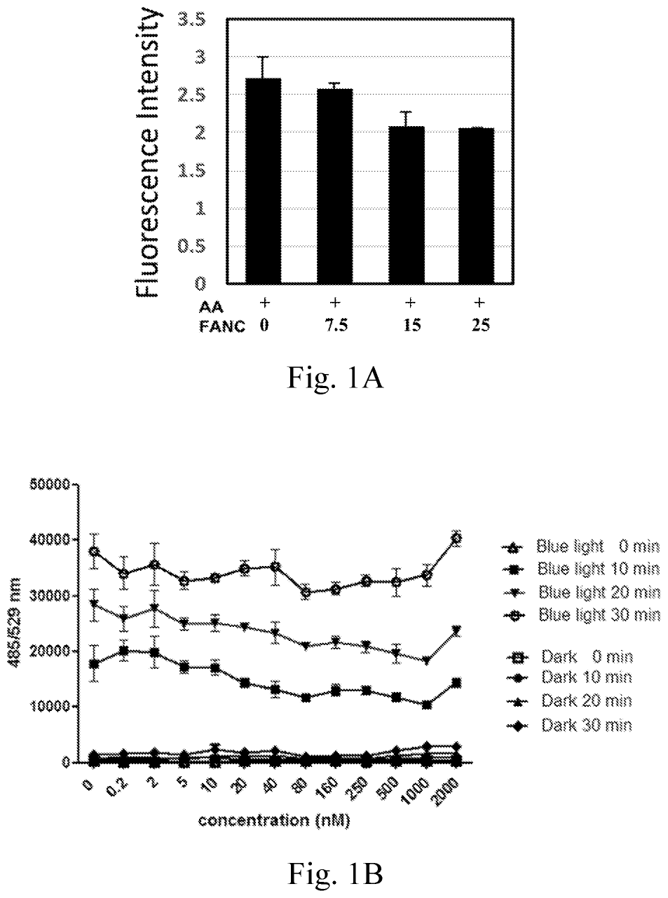

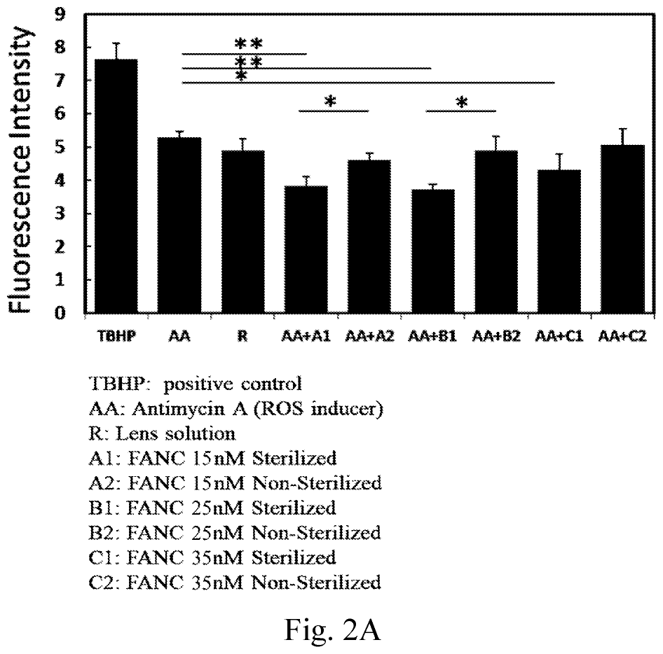

The anti-oxidative activity of the present DHLA coated gold nanoclusters was examined in this example. As the procedures described in Materials and Methods, the EPCs or BCEs were cultured with the DHLA coated gold nanoclusters followed by stimulating with AA or blue light irradiation. The intracellular ROS level was then determined by flow cytometry or spectrometer, and the results were respectively depicted in FIGS. 1A and 1B.

Compared with the control group (AA only), the administration of DHLA coated gold nanoparticles (FANC) reduced the ROS level in EPCs in a dose-dependent manner (FIG. 1A). The data of FIG. 1B further demonstrated that compared with the control group (i.e., the BCEs incubated in the dark without any irradiation), the irradiation of blue light time-dependently induced the ROS level in BCEs, and the treatment of DHLA coated gold nanoparticles decreased the intracellular ROS level stimulated by the blue light irradiation.

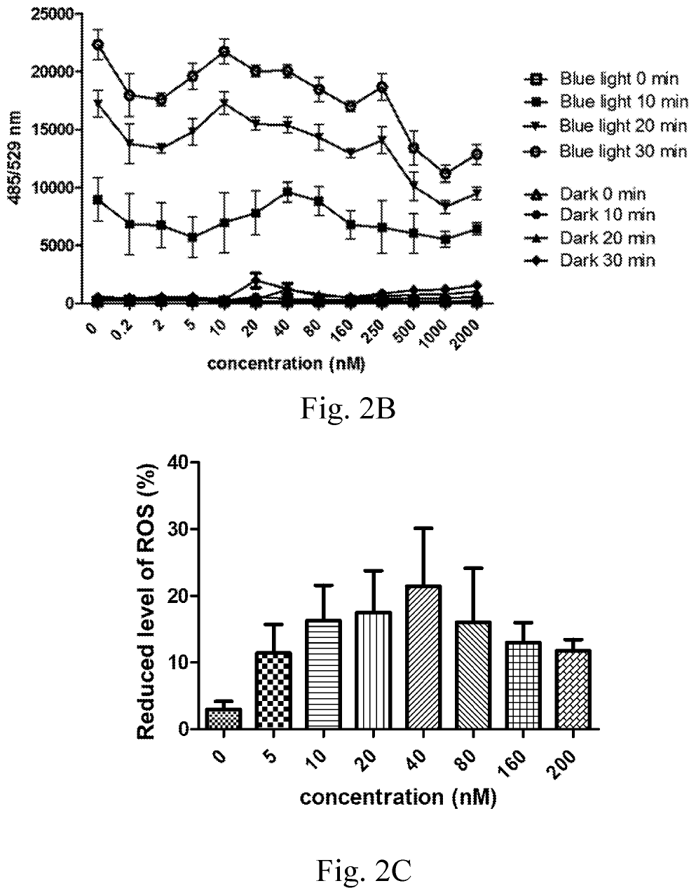

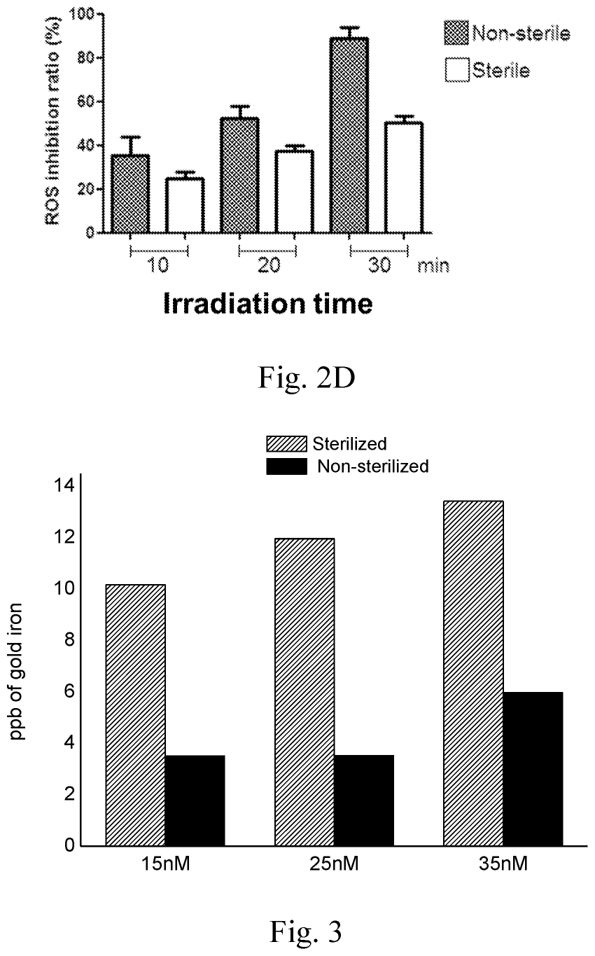

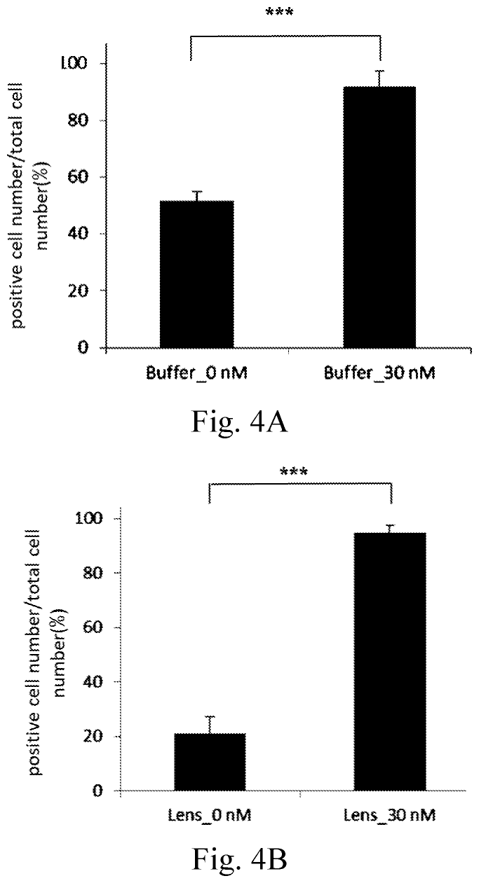

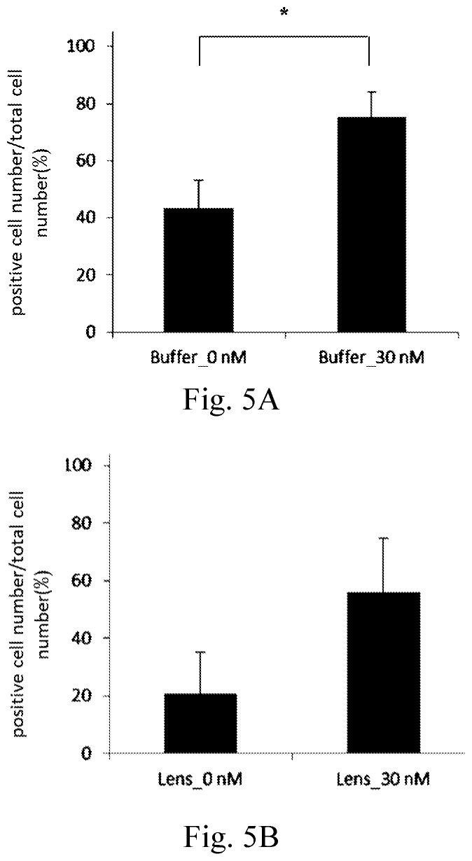

The DHLA coated gold nanoparticles were then sterilized under a pressure of 1.5 atm at 132° C. for 30 minutes as described in Materials and Methods, and incubated with EPCs or BCEs to examine their anti-oxidative activity. As the data of FIG. 2A depicted, sterilized DHLA coated gold nanoclusters exhibited higher anti-oxidative activity as compared to non-sterilized DHLA coated gold nanoclusters. It is further noted that the ROS level of cells treated with 15 nM or 25 nM sterilized DHLA coated gold nanoclusters was lower than that of 35 nM sterilized DHLA coated gold nanocluster treated cells (FIG. 2A). The result of FIG. 2B further confirmed the inhibitory effect of the present DHLA coated gold nanoclusters on intracellular ROS level, in which sterilized DHLA coated gold nanoclusters were capable of reducing the ROS level in BCEs at all tested concentrations (i.e., the concentrations from 0.2 to 2,000 nM). Compared to non-sterilized DHLA coated gold nanoclusters, sterilized DHLA coated gold nanoclusters also exhibited a superior anti-oxidative activity in BCEs, in which the ROS-inhibitory activity of sterilized DHLA coated gold nanoclusters was 10-30% higher than that of non-sterilized DHLA coated gold nanoclusters (FIGS. 2C and 2D).

It is evident from FIGS. 1 and 2 that the present DHLA coated gold nanoclusters, especially the DHLA coated gold nanoclusters pre-treated with sterilization process, are capable of reducing the ROS level in cells.

### Example 2 Absorption of DHLA Coated Gold Nanoclusters on Contact Lens

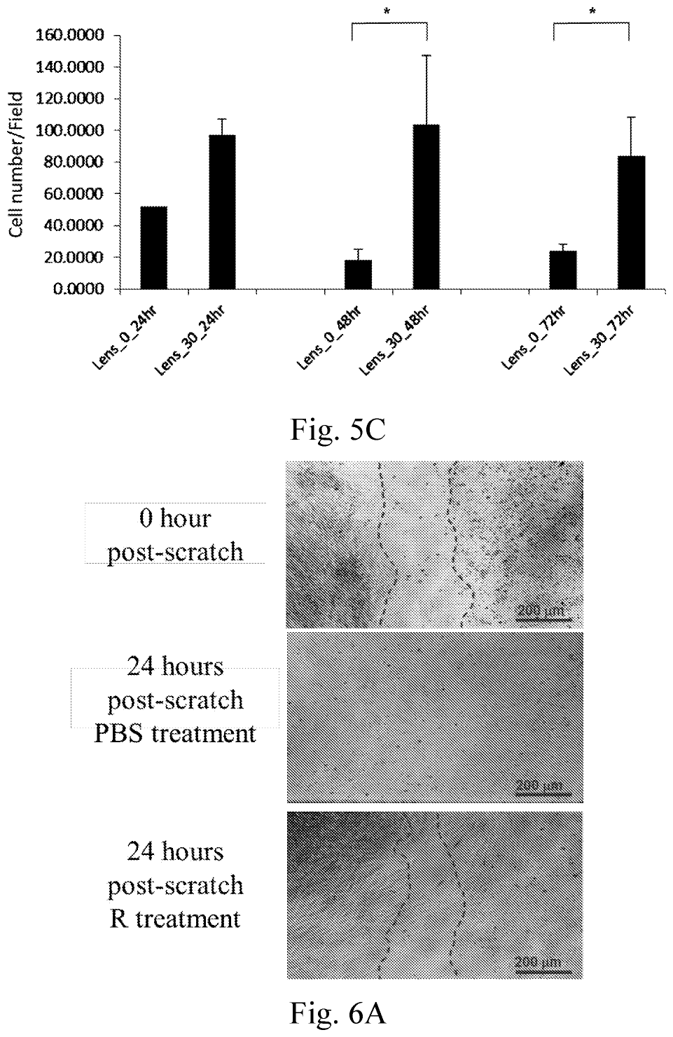

The absorption capability of the present DHLA coated gold nanocluster on a contact lens was evaluated in this example. The data of FIG. 3 indicated that after incubating at 132° C. for 30 minutes, the present DHLA coated gold nanoclusters at all tested concentrations (i.e., 15, 25 or 35 nM) could be efficiently absorbed to the contact lens. Of note, the absorption level of sterilized DHLA coated gold nanoclusters on contact lens was higher than that of non-sterilized DHLA coated gold nanoclusters.

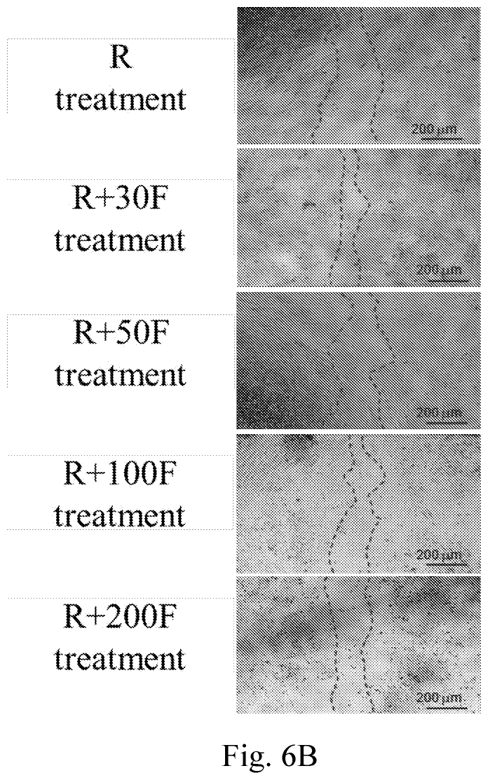

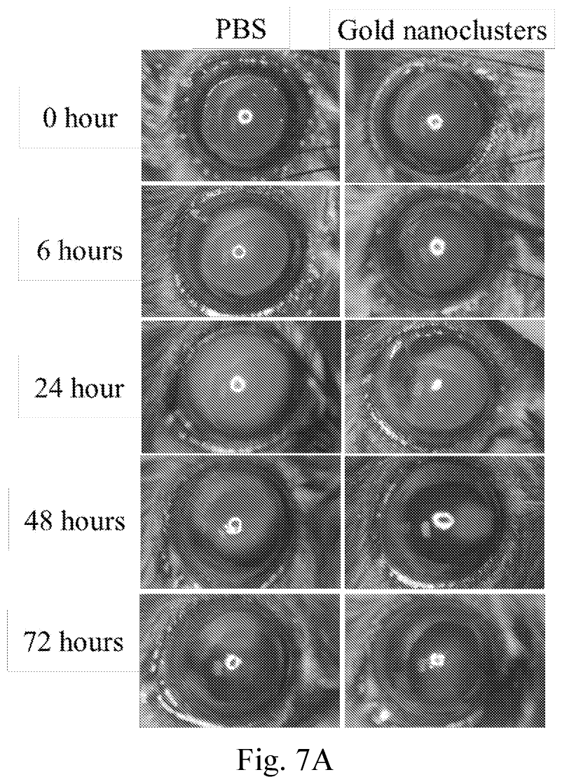

It is known that the blue light irradiation would cause cell damage via inducing oxidative stress thereby decreasing the expression level of GSH in cells. Accordingly, the GSH expression level in cornea cells administered with different treatments was measured so as to evaluate the anti-oxidative activity of the present DHLA coated gold nanoclusters. The results of FIGS. 4A and 4B indicated that both the medium containing 30 nM gold nanoclusters (Buffer_30 nM, FIG. 4A) and the contact lens pre-treated with 30 nM gold nanocluster (Lens_30 nM, FIG. 4B) significantly enhanced the intracellular GSH expression in the defense model. A similar result was observed in the repair model, in which the treatment of medium containing 30 nM gold nanoclusters (Buffer_30 nM, FIG. 5A) or the contact lens pre-treated with 30 nM gold nanocluster (Lens_30 nM, FIG. 5B) obviously increased the expression level of GSH as compared to the control groups (i.e., Buffer_0 nM, FIG. 5A; Lens_0 nM, FIG. 5B). The data of FIG. 5C further suggested that the protective effect of the present ocular lens may last for least 72 hours post-irradiation.

These results demonstrated that the contact lens having the present gold nanoclusters absorbed on its surface provides a protective effect on the cornea cells against ROS damage.

### Example 3 Therapeutic Effect of DHLA Coated Gold Nanoclusters

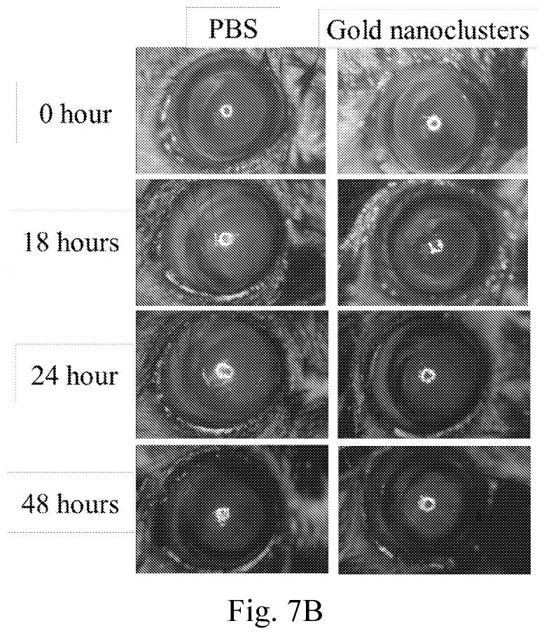

To investigate the effect of the present nanoclusters on corneal wound healing, rabbit cornea epithelial cells were scratched and treated by specified treatments as described in Materials and Methods. The data of Table 1 and FIG. 6A indicated that PBS-treated cornea cells exhibited marked wound closure 24 hours post-scratch; however, the treatment of eye drops (R treatment) reduced cell migration into a wound. The administration of gold nanoclusters (i.e., R+30F, R+50F, R+100F, or R+200F treatment) promoted the wound closure as compared to the eye drops treatment (R treatment) (Table 1 and FIG. 6B). These results suggested that the present DHLA coated gold nanoclusters was capable of promoting corneal wound healing.

TABLE 1
Percentage of Non-healing Area

| Treatment | Percentage (%) of Non-healing Area |
|---|---|
| Control | 24.22 |
| R | 18.62 |
| R + 30 F. | 8.18 |
| R + 50 F. | 17.02 |
| R + 100 F. | 11.35 |
| R + 200 F. | 14.13 |

* Control: 0 hour post-scratch; R: eye drops only; R + 30 F.: eye drops containing 30 nM DHLA coated gold nanoclusters; R + 50 F.: eye drops containing 50 nM DHLA coated gold nanoclusters; R + 100 F.: eye drops containing 100 nM DHLA coated gold nanoclusters; R + 200 F.: eye drops containing 200 nM DHLA coated gold nanoclusters.
** The percentage of non-healing area was calculated by image area of non-healing area/total area

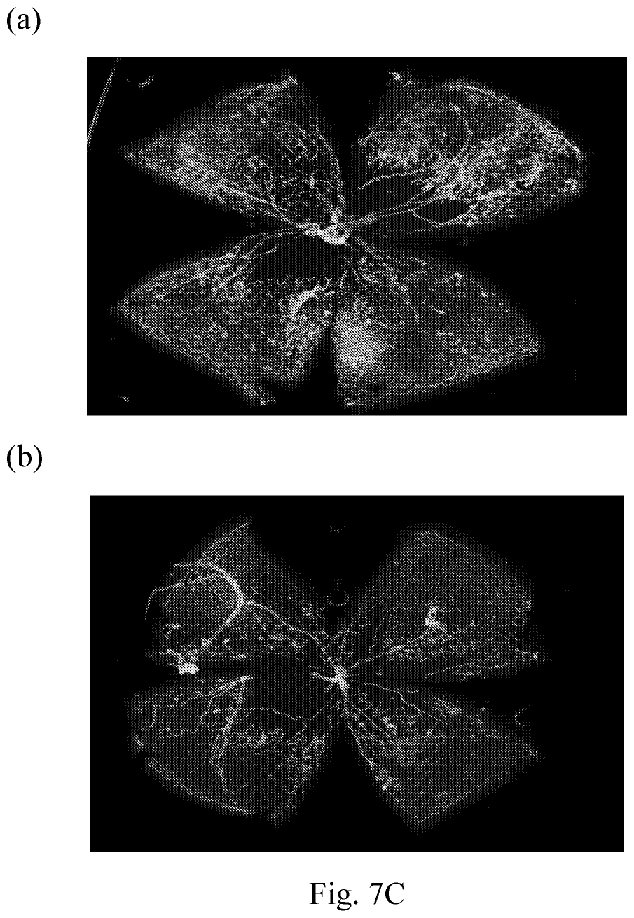

The effect of the present DHLA coated gold nanoclusters on promoting tissue repair was further confirmed by animal models, including the mechanical scrape model, the STZ-induced model, and the OIR model. The results of these models were respectively depicted in FIGS. 7A-7C. In the mechanical scrape model, the treatment of gold nanoclusters resulted in significantly smaller epithelial defect than that of the control group (i.e., the PBS treated mice) (FIG. 7A). According to the quantification analysis, the repairing percentages were 15.02% and 79.68% in mice treated with PBS and DHLA coated gold nanoclusters, respectively. A similar result was observed in the STZ-induced model, in which compared with the PBS treatment, the present DHLA coated gold nanoclusters exhibited better re-epithelialization (FIG. 7B). The repairing percentages in the PBS- and gold nanocluster-treated mice were respectively 4.85% and 51.18% 48 hours post-treatment. In the OIR model, a significant reduction in angiogenesis was observed in retina of the mice treated with the present DHLA coated gold nanoclusters (Panel (b) of FIG. 7C) compared to the mice treated with PBS control (Panel (a) of FIG. 7C).

These results suggested that the present DHLA coated gold nanoclusters are useful in promoting corneal repair and inhibiting pathological angiogenesis, and accordingly, may act as a potential agent to treat different ocular conditions.

In conclusion, the present disclosure provides a novel use of the DHLA coated gold nanoclusters in treating ocular conditions. According to examples of the present disclosure, the DHLA coated gold nanoclusters is useful in decreasing intracellular ROS level, promoting tissue repair (e.g., corneal repair), and inhibiting abnormal angiogenesis. Depending on desired purposes, the DHLA coated gold nanoclusters may be manufactured into an ocular lens or a pharmaceutical composition thereby providing a therapeutic benefit to a subject in need thereof.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. An ocular lens for treating an ocular condition in a subject, wherein the ocular lens is characterized in having a dihydrolipoic acid (DHLA) coated gold nanocluster absorbed thereon, wherein the DHLA coated gold nanocluster consists of, a gold nanocluster formed by a plurality of gold nanoparticles, and a plurality of DHLAs coated on the gold nanocluster.

2. The ocular lens of claim 1, wherein the DHLA coated gold nanocluster is pre-treated by a pressure of 1.2-2.0 atm at 120-140° C. for 10-60 minutes.

3. The ocular lens of claim 1, wherein the DHLA coated gold nanocluster has a particle size of 0.1 to 20 nm.

4. A method of preparing the ocular lens of claim 1, comprising,
subjecting the DHLA coated gold nanocluster solution to a pressure of 1.2-2.0 atm at 120-140° C. for 10-60 minutes; and
incubating a contact lens with a solution of the DHLA coated gold nanocluster having a concentration of 1-100 nM and a pH value of 7.5-9.0 at 15-50° C. for at least 30 minutes;
wherein the DHLA coated gold nanocluster has a particle size of 0.1 to 20 nm.

5. (canceled)

6. The method of claim 4, wherein the contact lens is incubated with 10-50 nM of the DHLA coated gold nanocluster solution.

7. (canceled)

8. A method of treating an ocular condition in a subject, comprising placing the ocular lens of claim 1 onto the cornea of the subject;
wherein the DHLA coated gold nanocluster is pre-treated by a pressure of 1.2-2.0 atm at 120-140° C. for 10-60 minutes;
wherein the DHLA coated gold nanocluster has a particle size of 0.1 to 20 nm.

9-10. (canceled)

11. The method of claim 8, wherein the ocular condition is associated with injury, reactive oxygen species (ROS) and/or angiogenesis.

12. The method of claim 11, wherein the ocular condition is eye injury, dry eye, conjunctivitis, uveitis, keratitis, retinitis, cataract, refractive error, glaucoma, optic neuropathy, macular degeneration, retinopathy, or retinitis pigmentosa.

13. A kit for treating an ocular condition, comprising,
a solution containing 1-100 nM of a DHLA coated gold nanocluster, wherein the DHLA coated gold nanocluster consists of, a gold nanocluster formed by a plurality of gold nanoparticles, and a plurality of DHLAs coated on the gold nanocluster, and
a contact lens immersed in the solution;
wherein the solution is pre-treated by a pressure of 1.2-2.0 atm at 120-140° C. for 10-60 minutes;
wherein the solution contains 10-50 nM of the DHLA coated gold nanocluster;
wherein the DHLA coated gold nanocluster has a particle size of 0.1 to 20 nm.

14-16. (canceled)

17. A pharmaceutical composition for treating an ocular condition in a subject, comprising a dihydrolipoic acid (DHLA) coated gold nanocluster, and a pharmaceutically acceptable excipient, wherein the DHLA coated gold nanocluster consists of, a gold nanocluster formed by a plurality of gold nanoparticles, and a plurality of DHLAs coated on the gold nanocluster, wherein the DHLA coated gold nanocluster has a particle size of 0.1 to 20 nm;
wherein the DHLA coated gold nanocluster is pre-treated by a pressure of 1.2-2.0 atm at 120-140° C. for 10-60 minutes.

18. (canceled)

19. A method of treating an ocular condition in a subject, comprising administering to the eye of the subject an effective amount of the pharmaceutical composition of claim 17;
wherein the DHLA coated gold nanocluster is pre-treated by a pressure of 1.2-2.0 atm at 120-140° C. for 10-60 minutes;
wherein the ocular condition is associated with injury, reactive oxygen species (ROS) and/or angiogenesis, and is eye injury, dry eye, conjunctivitis, uveitis, keratitis, retinitis, cataract, refractive error, glaucoma, optic neuropathy, macular degeneration, retinopathy, or retinitis pigmentosa.

20-22. (canceled)
